(19) United States
(12) Patent Application Publication
(10) Pub. No.: US 2011/0105538 A1
(43) Pub. Date: May 5, 2011

(54) DRUG RESPONSE MARKERS

(75) Inventors: Anthony M. Marinaki, London (GB); Jeremy David Sanderson, Kingston (GB); Bijay Kakkant Baburajan, Chelsfield (GB)

(73) Assignee: GUY'S & ST THOMAS'S NHS FOUNDATION TRUST, London (GB)

(21) Appl. No.: 12/921,929

(22) PCT Filed: Mar. 13, 2009
(86) PCT No.: PCT/GB09/00697

§ 371 Date: Dec. 10, 2010

(30) Foreign Application Priority Data
Mar. 13, 2008 (GB) 0804662.5
Mar. 14, 2008 (GB) 0804741.7

(51) Int. Cl. A61K 31/52; C12Q 1/68; A61P 1/00; A61P 29/00; A61P 35/02; A61P 25/00; A61P 19/02; A61P 11/06; A61P 3/10; A61P 17/06

(52) U.S. Cl. 514/263.3; 435/6

(57) ABSTRACT

The present invention relates to a method for predicting the response of a disease in a subject, preferably a human, to a drug providing 6-mercaptopurine, the method comprising the step of: (i) determining the presence or absence of a variant allele at a polymorphic site in a human leucocyte antigen (HLA)-G gene wherein the presence of said variant allele at said polymorphic site is indicative of clinical response or tolerance to said drug. The invention also provides a method of treating a subject suffering from a condition that would benefit from a drug providing 6-mercaptopurine.

### FIELD OF THE INVENTION

The invention relates to a method for predicting the response of a disease in a subject, preferably a human, to a drug providing 6-mercaptopurine and including a method of treating a subject suffering from a condition that would benefit from a drug providing 6-mercaptopurine.

### BACKGROUND TO THE INVENTION

The human leucocyte antigen region, located on the short arm of chromosome 6p codes the highly polymorphic, classical class I and II genes essential for normal lymphocyte function. It is clear from recent studies that the HLA region contains genes that determine susceptibility to chronic inflammatory bowel disease (ulcerative colitis, and Crohn's disease); that there are differences in associations between these two diseases; that ethnic groups differ and that some of these markers may have clinical significance in predicting disease course and the development of complications (1).

HLA-G is a nonclassical class I HLA which was first identified as molecule expressed by the foetal tissue cytotrophoblast at the foetal maternal interface (2). It has since then become evident that expression is not restricted to foetal cells, but also found in adult thymus, corneal epithelial cells, and the nail matrix in physiological conditions. These are considered to be immunologically privileged sites (3-5).

HLA-G differs from classical HLA by a low genomic polymorphism. 23 HLA-G alleles have been identified to date. The HLA-G primary transcript is alternatively spliced to form four membrane-bound forms (HLA G1, G2, G3 and G4) and three soluble protein isoforms (HLA G5,G6, and G-7). Other critical differences include the lack of allogenic reaction triggering and a deficiency in eliciting immune responses(6).

The foetus is semi-allogenic and a complex system of immune regulation prevents recognition by the mother's immune system. The expression of HLA G, at the foetal maternal interface suggests that this molecule allows for the maintenance of a viable foetus. This was attributed to a direct inhibitory effect of HLA-G on natural killer (NK) cells, which would otherwise kill targets devoid of HLA class 1 molecules. In addition, under pathological conditions, the molecule is expressed in inflammatory diseases, in infiltrating mononuclear cells within transplanted tissues and in tumour infiltrating antigen presenting cells. All this suggests a role for HLA-G in the regulation of immune responsiveness (7-12).

Inhibition of the immune system is a key strategy used by viruses to escape surveillance HLA-G is up regulated in monocytes and T-lymphocytes and of HIV patients and in patients with CMV infections (13, 14).

These data imply a rather non-specific effect on immune surveillance, which can manifest itself differentially, according to the tissue in which it is expressed. Hence conferring a survival benefit and a protective effect on a foetus, a virus or tumour cells.

HLA-G inhibits both the innate and adaptative immunity. Membrane-bound and soluble forms of HLA-G inhibit NK and CTL (cytotoxic T-lymphocyte) cytolysis (15-17). HLA G induces the differentiation of CD 4+ regulatory cells capable of inhibiting the reactivity of other T-cells. Furthermore, HLA-G, disrupts the maturation of dendritic cells leading to inhibition of cellular immune responses and prolonged graft survival. (18)

The inhibitory effects of HLA-G is mediated by inhibitory receptors called immunoglobulin like transcript (ILT-2 AND -4) and killer immunoglobulin like receptor (KIR) 2DL4, which expressed by immune cells. ILT2 is expressed by lymphoid and myeloid cells (19-21), ILT4 is expressed by myeloid cells only (22) and (KIR) 2DL4 is expressed by NK and some CD4+T-cells (23, 24). These receptors are up regulated at the cell surface when exposed to membrane-bound or soluble HLA-G. This promotes further immune tolerance. (25).

HLA-G exists in both membrane-bound and soluble isoforms. These are generated by alternatives splicing of the primary transcript. Four membrane forms (HLA-G 1, 2, 3, 4) and three secreted forms (HLA-G 5, 6, 7) are recognized. HLA-G 1 is the full-length form and is nearly identical to classical HLA class I antigens. It consists of three extra cellular domains, a transmembrane region and a cytoplasmic tail. It is assembled with 132 microglobulin linking a monopeptide to a heterotrimer. Like classical HLA molecules, HLA-G one can be cleaved from the cell surface and exist as soluble HLA G1 (sHLA G-1) in body fluids (26). The other membrane anchored HLA-G isoforms lack one or two alpha domains. These may also be shed from the cell surface, creating soluble HLA-G. In the soluble form the transmembrane and cytoplasmic domains are replaced by a short hydrophilic tail encoded by the 5′ sequences of intron 4(HLA-G 5, 6) or intron 2 (HLA-G 7) (26-30). This distinctive structural feature allows soluble HLA-G molecules to be differentiated from the proteolytically cleaved HLA-G molecules by a specific monoclonal polyclonal antibody recognizing the intron 4 or intron 2 sequence of HLA-G(31).

Decreased sHLA-G is observed in pre-eclampsia and preterm placental abruption both conditions associated with inadequate placental perfusion (32-34). It is associated with an improved pregnancy outcome in vitro fertilisation (IVF) and improved graft acceptance after cardiac and liver-kidney transplantation (7, 35). Impaired sHLA-G secretion has been reported in patients with ulcerative colitis, as compared to healthy subjects and Crohn's disease patients. This defect was related to an impairment in the IL-10 secretion in UC, but not in Crohn's disease (36). A detectable level of soluble HLA-G in a non-activated state is restricted to only a subset of individuals (7, 37). Monocytes may be the predominant cell type in the production of HLA-G5 in men and non-pregnant women.

The HLA-G protein is nearly monomorphic with only five amino acid polymorphisms (38). More are evident at the gene level (39). Exons 2, 3 and 4 polymorphism and their relevant amino acid substitutions form 14 alleles and a null allele (40-43). Altered expression of HLA G has been associated with HLA-G polymorphisms especially in the 3′ untranslated region (3′ UTR) and 5′ upstream regulatory region (5′ URR) of the gene. A 14 base pair sequence deletion/insertion polymorphism in the 3′ untranslated region has been described. HLA-G mRNA transcripts that include the 14 base pair sequence can be processed further without splicing of the first 92 base pairs of Exon 8(44, 45). This form is more stable than mRNA, which includes the 14 base pair sequence (46). The longer allele is also associated with lower levels of HLA-G mRNA (membrane-bound and soluble) and to some extent lower levels of soluble HLA-G (45).

In vitro studies have shown the influence of soluble HLA-G on cytokine expression of peripheral blood mononuclear cells(PBMC) and mixed lymphocyte cultures (MLC) (47).

More recently, there has been evidence suggesting a role for HLA-G in inflammatory bowel disease; more specifically, ulcerative colitis (UC). 34 patients with UC and 19 patients with CD were studied by immunohisto-chemistry for HLA-G and IL-10. All patients with UC (n=24), expressed HLA-G at the apical surface of the intestinal epithelial cells (IEC), and the crypts of Lieberkuhn. This was observed in both non inflamed and inflamed areas. No expression and subsequent staining was observed in any patients with Crohn's disease. There was a corresponding increase in lamina propria IL-10 expression in all the patients expressing HLA-G. The monoclonal antibody used recognize denatured HLA-G isoforms (48).

IECs are important in innate and adaptative immunity. HLA-G is important in immune tolerance, and it is possible that the selective expression of HLA-G in the cells influences tolerance to food antigens and gut microflora. Furthermore, these molecules may protect IECs from NK mediated cell lysis. One group has demonstrated that PBMC culture of decidual cells with cells expressing HLA-G leads to a shift from TH1 to TH2 cytokine expression (reduced TNF-α and IFN-γ and increased IL-4 (49). This, in conjunction with the findings of Torres et al suggest a role in inflammatory bowel disease.

In contrast to these findings, another group has demonstrated a clear difference in the production of sHLA-G molecules in IBD patients. Here, LPS-stimulated PBMCs from UC patients produced lower sHLA-G levels in association with lower IL-10 production. On the other hand, in Crohn's disease, the production of both these molecules is increased. While it is possible that the recognized lack of IL-10 production in UC and increased levels of the cytokine in Crohn's disease may be the cause for this discrepancy, it is proposed that estimation of soluble HLA-G may have a diagnostic role in inflammatory bowel disease(36).

Soluble HLA-G is a component of the human immune system which promotes immune tolerance. Its gene is subject to a common 14 base pair insertion/deletion, the presence of which has been extensively studied due to its association with pregnancy loss. Presence of the deletion polymorphism confers a favourable outcome.

The location of the polymorphism is set out in Rousseau, P. et al., Human Immunology 64, 1005-1010 (2003), the contents of which are hereby incorporated by reference. The insertion occurs in the 3′ untranslated region of Exon 8, the 14 base pair sequence of which is given in FIG. 1 of Rousseau et al., together with its flanking regions.

The correlation of the presence or absence of the 14 bp polymorphism is described in: V. Rebmann1, K. van der Ven, et al., Tissue Antigens, Volume 57 Issue 1 Page 15-21, January 2001, “Association of soluble HLA-G plasma levels with HLA-G alleles”.

Following on from this work, investigators in the field of rheumatoid arthritis discovered that this genetic polymorphism was also associated with a lack of response to the immunomodulating drug methotrexate (Baricordi O R, Govoni M, Rizzo R, Trotta F. In rheumatoid arthritis, a polymorphism in the HLA-G gene concurs in the clinical response to methotrexate treatment. Ann Rheum Dis 2007; 66(8):1125-1126 and Rizzo R, Rubini M, Govoni M et al. HLA-G 14-bp polymorphism regulates the methotrexate response in rheumatoid arthritis. Pharmacogenet Genomics 2006; 16(9):615-623). Again it is the deletion polymorphism that is associated with a favourable response.

The inventors then examined the association between this same ins/del polymorphism and response to methotrexate in inflammatory bowel disease and confirmed that it also predicts non-response to methotrexate in this disease background. This finding was published in abstract form for the 2007 American Gastro Association meeting. (Baburajan et al. 2007 Gastroenterology, vol. 132, April Supplement 1)

In view of the foregoing there is a need to determine whether this polymorphism is associated with response to other immunomodulators. This information will provide a powerful tool in determining the effectiveness of these drugs in treating certain conditions.

The present inventors have also surprisingly found that the aforementioned deletion/insertion polymorphism, together with other newly-reported markers of non-response to the drug, azathioprine, provide a powerful predictor of the expected response of a patient to this and other related drugs.

### STATEMENTS OF THE INVENTION

According to a first aspect of the invention there is provided a method for predicting the response of a disease, in a subject, to a drug providing 6-mercaptopurine said method comprising the step of:
(i) determining the presence or absence of a variant allele at a polymorphic site in a human leucocyte antigen (HLA)-G gene wherein the presence of said variant allele at said polymorphic site is indicative of clinical response or tolerance to said drug.

In a preferred method the polymorphic site is within the 3′ untranslated region of the HLA-G gene. Preferably the polymorphic site is exon 8.

In a preferred method of the invention the variant allele is a 14 base pair insertion or deletion polymorphism. The 14base pair insertion/deletion polymorphism is preferably that represented by the sequence shown in FIG. 1.

In a method of the invention the variant allele is preferably a 14 base pair deletion polymorphism, for example the variant allele is of G*010101, G*0102 and/or G*010401.

In an alternative method of the invention the variant allele is a 14 base pair insertion polymorphism, for example the variant allele is of G*010102, G*010103, G*0103, G*0105N and/or G*0106.

In a method of the invention the presence of the insertion polymorphism may be indicative of an expected less favourable clinical response of the disease to said drug.

As used herein “less favourable clinical response” means either non responsive to the drug or a reduced response to the drug when compared to the expected “normal” response to the drug demonstrated by a subject.

In a method of the invention the presence of the deletion polymorphism may be indicative of an expected favourable clinical response of the disease to said drug.

The term “polymorphism” refers to the coexistence of multiple forms of a sequence.

Thus, a polymorphic site is the location at which sequence divergence occurs. The different forms of the sequence which exist as a result of the presence of a polymorphism are referred to as “alleles”. Examples of the ways in which polymorphisms are manifested include restriction fragment length polymorphisms (Botstein et al Am J Hum Genet. 32 314-331 (1980)), variable number of tandem repeats, hypervariable regions, minisatellites, di- or multi-nucleotide repeats, insertion elements and nucleotide or amino acid deletions, additions or substitutions. A polymorphic site may be as small as one base pair, which may alter a codon thus resulting in a change in the encoded amino acid sequence.

Single nucleotide polymorphisms arise due to the substitution, deletion or insertion of a nucleotide residue at a polymorphic site. Such variations are referred to as SNPs. SNPs may occur in protein coding regions, in which case different polymorphic forms of the sequence may give rise to variant protein sequences. Other SNPs may occur in non-coding regions. In either case, SNPs may result in defective proteins or regulation of genes, thus resulting in disease. Other SNPs may have no phenotypic effects, but may show linkage to disease states, thus serving as markers for disease.

The method of the invention may be performed by genotyping and includes any method of identifying differences between nucleic acid sequences. A number of suitable methods are available in the art and include, but are not limited to, direct probing, allele specific hybridisation, PCR, Allele Specific Amplification (ASA) (WO93/22456), Allele

Specific Hybridisation, single base extension (U.S. Pat. No. 4,656,127), ARMS-PCR, Taqman (U.S. Pat. No. 4,683,202; 4,683,195; and 4,965,188), oligo ligation assays, single-strand conformational analysis ((SSCP) Orita et al PNAS 86 2766-2770 (1989)), Genetic Bit Analysis (WO 92/15712) and RFLP direct sequencing, mass-spectrometry (MALDI-TOF) and DNA arrays.

The above described methods may require amplification of a DNA sample from the subject, and this can be done by techniques known in the art such as PCR (see PCR Technology: Principles and Applications for DNA Amplification (ed. H. A. Erlich, Freeman Press, NY 1992; PCR Protocols: A Guide to methods and Applications (eds. Innis et al., Academic press, San Diego, Calif. 1990); Mattila et al., Nucleic Acids Res. 19 4967 (1991); Eckert et al., PCR Methods and Applications 117 (1991) and U.S. Pat. No. 4,683,202. Other suitable amplification methods include ligase chain reaction (LCR) (Wu et al., Genomics 4 560 (1989); Landegran et al., Science 241 1077 (1988)), transcription amplification (Kwoh et al., Proc Natl Acad Sci USA 86 1173 (1989)), self sustained sequence replication (Guatelli et al., Proc Natl Acad Sci USA 87 1874 (1990)) and nucleic acid based sequence amplification (NASBA). The latter two methods both involve isothermal reactions based on isothermal transcription which produce both single stranded RNA and double stranded DNA as the amplification products, in a ratio of 30 or 100 to 1, respectively. Where it is desirable to analyse multiple samples simultaneously, it may be preferable to use arrays as described in WO95/11995. The array may contain a number of probes, each designed to identify

Thus in a preferred aspect of the invention the method comprises the steps of:
(i) genotyping said subject at a polymorphic site in a human leucocyte antigen (HLA)-G gene; and
(ii) determining the presence or absence of a variant allele at a polymorphic site in a human leucocyte antigen (HLA)-G gene wherein the presence of said variant allele at said polymorphic site is indicative of clinical response or tolerance to said drug.

The methods of the invention are preferably carried out on a sample removed from a subject. Any biological sample comprising cells containing nucleic acid is suitable for this purpose. Examples of suitable samples include whole blood, leukocytes, semen, saliva, tears, buccal, skin or hair. For analysis of cDNA, mRNA or protein, the sample must come from a tissue in which the sequence of interest is expressed. Blood is a readily accessible sample. Thus, the method of the invention preferably includes the steps of obtaining a sample from a subject, and preparing nucleic acid from the sample.

In an alternative method of the invention the presence of absence of the deletion-insertion polymorphism is determined by determining the concentration of soluble HLA-G (sHLA-G) in a sample from the subject; decreased levels of sHLA-G being indicative of the presence of the insertion variant. In particular, decreased levels of sHLA-G by comparison to expected population distribution of levels of sHLA-G in populations having the deletion variant, being indicative of the presence of the insertion variant.

Methods for detecting the presence or absence, or concentration of sHLA-G are given in: V. Rebmann, J. LeMaoult, N. Rouas-Freiss, E. D. Carosella, H. Grosse-Wilde (2007) “Quantification and identification of soluble HLA-G isoforms”, Tissue Antigens 69 (s1), 143-149 (the contents of which are hereby incorporated by reference) and elsewhere.

The metabolic conversion of thiopurine drugs to purine nucleotides and the subsequent incorporation of these derivatives into DNA plays an important role in both the efficacy and toxicity of these drugs. A catabolic route for thiopurine metabolism is catalysed by the enzyme thiopurine methyltransferase (TPMT) which inactivates thiopurines by converting them to thiomethyl derivatives such as 6-methylmercaptopurines (6-MP). A balance must be established to ensure that sufficient drug is converted to the nucleotide to act as an antimetabolite but that the level of antimetabolite does not become so high as to cause potentially harmful bone marrow suppression. Patients with low TPMT activity are at risk of fatal haematological toxicity in response to 6-MP therapy.

Thus a preferred method of the invention further comprises the step of determining the presence or absence of a variant allele at a polymorphic site within a thiopurine methyltransferase (TPMT) gene wherein the presence of said variant allele at said polymorphic site is indicative of a decreased clinical tolerance, or clinical intolerance, to said drug.

As used herein the term “tolerance” refers to the capacity of the body to endure a drug without an adverse drug reaction. In certain instances “tolerance” means non-responsiveness to said drug.

The presence or absence of a variant allele at a polymorphic site within a TPMT gene may be determined by measuring TPMT activity in a biological sample from said subject. Either an absence, or lower than normal levels, of TPMT activity may be indicative of a decreased tolerance to said drug.

Methods for determining TPMT activity are known in the art. For example since TPMT specifically catalyses the methylation of thiopurine substrates to produce methylated thiopurine reaction products, the rate of formation of methylated thiopurine reaction products is a reliable indicator of TPMT activity in sample from a subject—see US2006/0263840.

Alternatively the presence or absence of a variant allele at a polymorphic site within a TPMT gene may be determined by genotyping TPMT. Thus the method of the invention may further comprise the steps of
(i) genotyping said subject at a polymorphic site in a TPMT gene; and
ii) determining the presence or absence of a variant allele at said polymorphic site wherein the presence of said variant allele at said polymorphic site is indicative of a decreased clinical tolerance, or clinical intolerance, to said drug. The presence of a variant allele(s) at said polymorphic site may be indicative of a decreased tolerance to said drug. In this case, an alternative drug should be considered.

SNP polymorphisms resulting in low TPMT activity include polymorphisms within the alleles TPMT1, TPMT3A, TPMT3B, TPMT3C, TPMT3D, TPMT4, TPMT5, TPMT6, TPMT7 and TPMT8. The SNP polymorphism may be within allele TPMT3. For example polymorphism of allele TPMT3 (e.g. TPMT3A) is at residue 460 wherein G is replaced by A. By way of a further example the polymorphism of allele TPMT (e.g. TPMT3A) is at residue 719 wherein A is replaced by G.

In a preferred aspect of the invention the method further comprises the step of determining the presence or absence of a variant allele at a polymorphic site in an aldehyde oxidase (AOX) gene wherein the presence of said variant allele at said polymorphic site is indicative of a clinical response to said drug.

The presence or absence of a variant allele at a polymorphic site within an AOX gene may be determined by measuring AOX activity in a biological sample from said subject, see for example Human liver aldehyde oxidase: inhibition by 239 drugs. Obach R S, Huynh P, Allen M C, Beedham C. J Clin Pharmacol. 2004 January; 44(1):7-19 Drug-metabolizing ability of molybdenum hydroxylases. Kitamura S, Sugihara K, Ohta S. Drug Metab harmacokinet. 2006 April; 21(2):83-98.

Alternatively the presence or absence of a variant allele at a polymorphic site within an AOX gene may be determined by genotyping AOX. Thus the method of the invention may further comprise the steps of
(i) genotyping said subject at a polymorphic site in an AOX gene; and
ii) determining the presence or absence of a variant allele at said polymorphic site wherein the presence of said variant allele at said polymorphic site is indicative of a clinical response to said drug.

In a preferred method the polymorphic site in an AOX gene is in an aldehyde oxidase 1 gene. The polymorphic site may be 3404A. Typically the said variant allele is a SNP polymorphism for example a nucleotide residue other than A. The SNP polymorphism may be G namely 3404 A to G.

The presence of said variant allele at a polymorphic site in an AOX gene may be indicative of a less favourable clinical response to said drug.

A preferred aspect of the invention provides a method for predicting the response of a disease, in a subject, to a drug providing 6-mercaptopurine said method comprising the step of determining the presence or absence of a variant allele at a polymorphic site in a HLA-G gene and an AOX gene wherein the presence of said variant allele at said polymorphic sites is indicative of clinical response or tolerance to said drug.

In a further preferred aspect the method comprises the steps:
(i) genotyping said subject at a polymorphic site in a human leucocyte antigen (HLA)-G gene and an aldehyde oxidase gene; and
(ii) determining the presence or absence of a variant allele at a polymorphic site in a HLA-G gene and an AOX gene wherein the presence of said variant allele at said polymorphic sites is indicative of clinical response or tolerance to said drug.

In a yet further preferred aspect the method comprises the steps:
(ii) genotyping said subject at a polymorphic site in a human leucocyte antigen (HLA)-G gene, an aldehyde oxidase gene and a TPMT gene; and
(ii) determining the presence or absence of a variant allele at a polymorphic site in a HLA-G gene, an AOX gene and a TPMT gene wherein the presence of said variant allele at said polymorphic sites is indicative of clinical response or tolerance to said drug.

As used herein the term “drug providing 6-mercaptopurine” may include a drug selected from the group consisting of 6-mercaptopurine, azathioprine, 6-thioguanine and 6-methyl-mercaptopurine riboside.

As used herein, the term “6-mercaptopurine” includes any drug that can be metabolised to an active 6-mercaptopurine metabolite that has therapeutic efficacy such as 6-thioguanine. Exemplary 6-mercaptopurine drugs as defined herein include 6-mercaptopurine and azathioprine. Both of 6-mercaptopurine and azathioprine can be metabolised to 6-mercaptopurine metabolites such as 6-thioguanine, 6-methyl-mercaptopurine and 6-thiouric acid. Other 6-mercaptopurine drugs include, for example, 6-methylmercaptopurine riboside and 6-thioguanine.

As used herein, the term “6-thioguanine” includes 6-thioguanine or analogues thereof, including molecules having the same base structure, for example 6-thioguanine ribonucleoside, 6-thioguanine ribonucleotide mono, di and tri-phosphate, 6-thioguanine deoxyribonucleoside and 6-thioguanine deoxyribonucleotide mono, di, and triphosphate. The term 6-thioguanine also includes derivatives of 6-thioguanine, including chemical modifications of 6-thioguanine, so long as the structure of the 6-thioguanine is preserved.

As used herein, the term “6-methyl-mercaptopurine” includes 6-methyl-mercaptopurine or analogues thereof, including analogues having the same base structure, for example, 6-methyl-mercaptopurine ribonucleoside, 6-methyl-mercaptopurine ribonucleotide mono-, di-, and tri-phosphate, 6-methyl-mercaptopurine deoxyribonucleoside, and 6-methyl-mercaptopurine deoxyribonucleotide mono-, di- and tri-phosphate. The term 6-methyl-mercaptopurine also includes derivatives of 6-methyl-mercaptopurine, including chemical modifications of 6-methyl-mercaptopurine, so long as the structure of the 6-methyl-mercaptopurine is preserved.

In one embodiment of the invention the drug is 6-mercaptopurine.

In a further embodiment of the invention the drug is 6-thioguanine.

Preferably the drug is azathioprine.

The subject may be any animal, preferably a mammal, and more preferably human.

A further aspect of the invention provides a method of treating or preventing disease in a subject comprising the steps of:
(i) determining the presence or absence of a variant allele at a polymorphic site in a human leucocyte antigen (HLA)-G gene; and
(ii) if said variant allele is present, administering a drug providing 6-mercaptopurine in order to prevent, delay or reduce the disease.

In a preferred method said variant allele is the presence of the 14 base pair deletion polymorphism as described hereinbefore.

A further preferred method of treatment includes determining the presence or absence of a variant allele at a polymorphic site in an AOX gene.

A yet further preferred method of treatment includes determining the presence or absence of a variant allele at a polymorphic site in a TPMT gene.

In a preferred method of the invention the disease is a disease exhibiting variable response to said drug for example azathioprine, 6-mercaptopurine or 6-thioguanine.

The disease to be treated may be an immune disorder for example a disorder selected from an immune-mediated gastrointestinal disorder, an autoimmune disorder and graft versus host disease.

The immune disorder may be an inflammatory disorder for example selected from atopy, asthma or psoriasis or type II diabetes

The immune disorder may be an inflammatory joint disease such as rheumatoid arthritis, multiple sclerosis and alkylating spondylitis.

The immune disorder may be leukaemia, for example lymphoblastic leukaemia.

Where the immune disorder is an immune-mediated gastrointestinal disorder the disorder may be selected from the group consisting of inflammatory bowel diseases for example Ulcerative colitis and Crohn's disease, lymphocytic colitis, microscopic colitis, colaagenous colitis, autoimmune enteropathy, allergic gastrointestinal disease and eosinophilic gastrointestinal disease.

A further aspect of the invention provides a kit comprising a means for determining the presence or absence of a variant allele at a polymorphic site in a HLA-G gene wherein the presence of said variant allele at said polymorphic site is indicative of a clinical response or tolerance to a drug providing 6-mercaptopurine.

The kit may comprise the components necessary to determine the presence or absence of said variant allele including PCR primers (such as allele specific primers) and/or probes, PCR enzymes, restriction enzymes, and DNA or RNA purification means. The kit may contain at least one pair of primers, or probes. Primers may be selected from the group consisting of:

5′-AGCTTCACAAGAATGAGGTGGAGC-3′
5′-AATGAGTCCGGGTGGGTGAGCGA-3′

Other components include labeling means, buffers for the reactions. In addition, a control nucleic acid sample may be included, which comprises a wild type or variant nucleic acid sequence as defined above, or a PCR product of the same. The kit will usually also comprise instructions for carrying out the method of the invention and a key detailing the correlation between the results and the likely outcome of therapy with said drug. The kit may also comprise said drug.

The invention may also comprise any of the following concepts:

A method of determining the expected response of a disease, in a human subject, to azathioprine comprising the step of determining the presence or absence of the 14base pair deletion-insertion polymorphism described herein (see Rousseau et al, Human Immunology, 1005-1010, 2003, especially FIG. 1); the presence of the insertion variant being indicative of an expected less favourable response of the disease to azathioprine.

A method of determining the expected response of disease, in a human subject, to 6-mercaptopurine comprising the step of determining the presence or absence of the 14 bp deletion-insertion polymorphism described herein (see Rousseau et al, Human Immunology, 1005-1010, 2003, especially FIG. 1); the presence of the insertion variant being indicative of an expected less favourable response of the disease to 6-mercaptopurine.

A method of determining the expected response of a disease, in a human subject, to 6-thioguanine comprising the step of determining the presence or absence of the 14 bp deletion-insertion polymorphism described herein (see Rousseau et al, Human Immunology, 1005-1010, 2003, especially FIG. 1); the presence of the insertion variant being indicative of an expected less favourable response of the disease to 6-thioguanine.

A method according to any of these preceding concepts, wherein the presence of absence of the deletion-insertion polymorphism is determined by determining the concentration of soluble HLA-G (sHLA-G) in a sample from a subject; decreased levels of sHLA-G being indicative of the presence of the insertion variant. In particular, decreased levels of sHLA-G by comparison to expected population distribution of levels of sHLA-G in populations having the deletion variant, being indicative of the presence of the insertion variant.

Methods for detecting the presence or absence, or concentration of sHLA-G are given in: V. Rebmann, J. LeMaoult, N. Rouas-Freiss, E. D. Carosella, H. Grosse-Wilde (2007) “Quantification and identification of soluble HLA-G isoforms”, Tissue Antigens 69 (s1), 143-149 (the contents of which are hereby incorporated by reference) and elsewhere.

A method according to any of these preceding concepts, wherein the disease is a disease exhibiting variable response to the pharmaceutical agent (azathioprine, 6-mercaptopurine or 6-thioguanine).

A method according to any of these preceding concepts wherein the disease is an autoimmune disorder.

A method according to any of these preceding concepts wherein the disease is inflammatory bowel disease.

A method according to any of these preceding concepts wherein the disease is acute lymphoblastic leukaemia.

A method according to any of these preceding concepts wherein the disease is a disease, or condition, responsive to immunosuppressant therapy, such as organ rejection following organ transplantation.

A method of determining the expected response of a disease, in a human subject, to azathioprine comprising the steps of:
determining the presence or absence of “The TPMT Marker”;
determining the presence or absence of the “HLA-G Marker”;
determining the presence or absence of the “(SNP AOX3404A>G) Marker”; the presence of a plurality of such markers, each indicating an expected less favourable response of the disease to azathioprine, being indicative of a less favourable response of the disease to azathioprine.

A method according to the preceding concept wherein the plurality of such markers includes the “(SNP AOX3404A>G) Marker”.

A method according to either preceding concept wherein the plurality of such markers includes the “HLA-G Marker”.

A method wherein all three markers are present.

These methods may also be used for determining the expected response to 6-mercaptopurine or 6-thioguanine, and therefore the inventive concepts also include:

Methods of determining the expected response of a disease, in a human subject, to a medicament selected from:
6-mercaptopurine;
6-thioguanine; comprising any of the preceding methods mutatis mutandis.

In particularly preferred embodiments of any these methods disclosed herein, the disease is selected from the group comprising:
an autoimmune disorder;
inflammatory bowel disease;
acute lymphoblastic leukaemia;
a disease, or condition, responsive to immunosuppressant therapy, such as organ rejection following organ transplantation.

The inventive concept also includes test kits adapted to perform any of these methods.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

FIG. 1 (a) the sequence of the 14 base pair insertion/deletion polymorphism (SNP number rs1704) in exon 8 of HLA-G gene; (b) location of the SNP, rs1704;

FIG. 2: Photograph of a 2.5% agarose gel (Invitrogen, Paisley, Scotland, UK) after electrophoresis and staining with ethidium bromide (0.5 μg/ml)

FIG. 3: A histogram of genotype frequencies of the HLA-G 14 bp ins/del in patients treated with Methotrexate

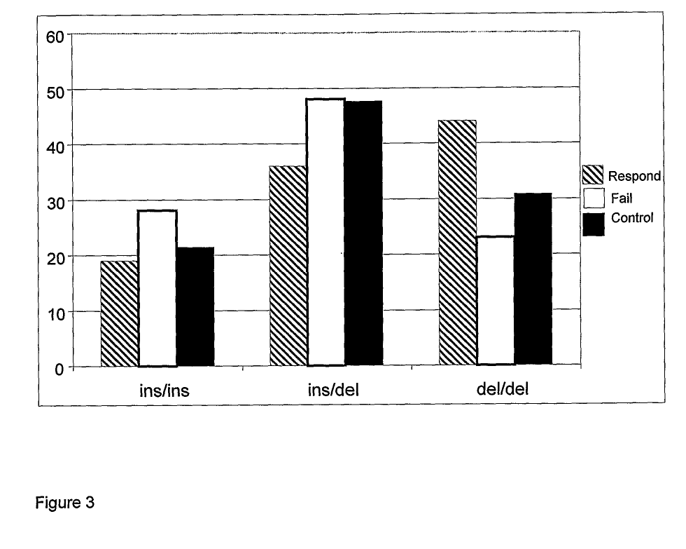

FIG. 3 4: A histogram showing response of patients to azothioprine

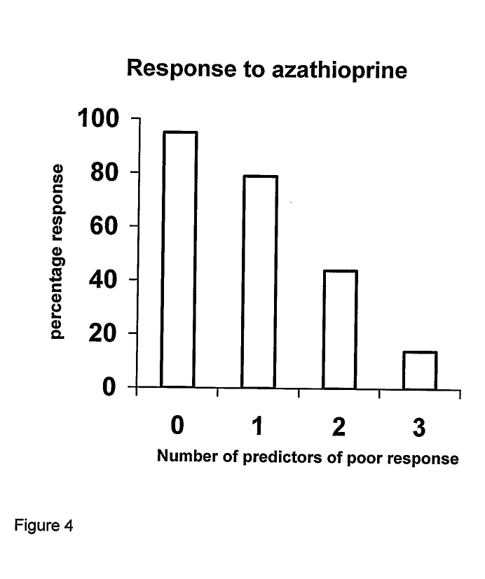

### EXAMPLE 1

### Materials and Methods

### Techniques

Venous blood for DNA extraction was collected in EDTA tubes and stored at −70° C. Genomic DNA was isolated from whole blood using standard techniques. DNA was amplified with recombinant Taq DNA polymerase recombinant (Invitrogen Ltd, Paisley, UK) in a total volume of 20 μl with 0.5 μM of each primer. Reactions were supplemented with 5% DMSO.

HLA-G 14 by ins/del

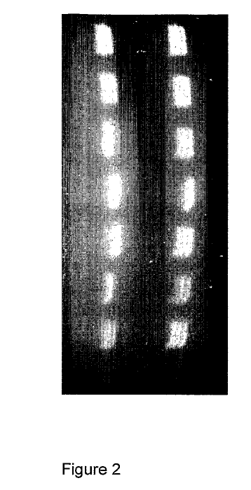

The HLA-G 14-bp polymorphism was genotyped by polymerase chain reaction. 2 μg of genomic DNA was amplified in a 25 μl reaction, with a final concentration of the following reagents: reaction buffer (Roche, Basel, Switzerland) 1×; each deoxynucleoside triphosphate (Roche) 0.2 mmol/l; MgCl₂ (Roche) 1.5 mmol/l; Taq Polymerase (Roche) 0.75 units; and each primer (5 KBHLAG, 5′-AGCTTCACAAGAATGAGGTGGAGC-3′ and PROHLAG3,5′-AATGAGTCCGGGTGGGTGAGCGA-3′). The thermocycling conditions were 94° C. for 2 min, 25 cycles of 94° C. for 30 s, 64° C. for 60 s, and 72° C. for 2 min, followed by 72° C. for 10 min. The amplified products were visualized by electrophoresis on a 2.5% agarose gel (Invitrogen, Paisley, Scotland, UK) containing ethidium bromide (0.5 μg/ml) (FIG. 2)

### The Study Population

### Methotrexate

Patients were recruited from gastroenterology outpatient clinics at Guy's and St

Thomas's Hospital, London; Addenbrooke's Hospital, Cambridge and the John Radcliffe Hospital, Oxford. After informed consent was obtained, casenotes were retrospectively reviewed by two separate reviewers and phenotypic and drug response/toxicity data was extracted. Blood samples were collected and stored for analysis. Patients were classified into responders and non-responders, based on inability to withdraw from steroid therapy or achieve an adequate therapeutic response. Patients needed to have received a minimum of three months of methotrexate therapy before decisions on efficacy could be made. Hepatotoxicity was defined as an elevation and liver enzymes persistently to more than three times the upper limit of normal. None of the patients in the study group have had liver biopsies.

### Azathioprine

The azathioprine cohort was prospectively recruited at Guy's and St Thomas's Hospital, London, over a period of two years. After informed consent, phenotypic and drug response data was collected prospectively and correlated with genotype.

### Statistical Methods

The Instat Graph pad programme was used to calculate, Fisher's exact test and odds ratios which are reported in this study

### Results

### Methotrexate Group

A total of 204 patients were included in to the study. 99 were female (49%). 135 (66%) patients had Crohn's disease, and 34 (16%) had ulcerative colitis. 33 (16%) patients were classified as indeterminate colitis or data was unavailable. In the Crohn's disease and group, 29 (18%) had isolated colonic involvement, 70 (25%) had ileo colonic involvement, 17 (10%) had isolated ileal involvement and four had distal disease affecting the rectum of anal canal. 37 (24%) patients had perianal disease with fistulae. In the ulcerative colitis group 15 (53%) had left-sided colitis, five (18%) had isolated proctitis and eight (29%) had pancolitis.

The mean age at diagnosis in the Crohn's disease group among those treated with methotrexate was 29 with a range of 9 to 78 years. In the ulcerative colitis group, the average age was 38 years, with a range of 19 to 75.

Extra intestinal disease manifestations were noted in a significant number of patients in the Crohn's disease group (37%). 39 patients had polyarthritis six had erythema nodosum and 10 had some form of ocular involvement. Only two had documented iritis. In the ulcerative colitis group 7 patients had arthritis, 1 had erythema nodosum. No other extraintestinal manifestations were noted.

Indication for in immune modulation, were active disease (either not responsive to or not tolerant of steroids) in 31% patients; steroid dependent disease in 54% patients or fistulising disease in 21% Of patients.

MTX was used for failure of azathioprine to achieve a therapeutic response in 53 patients and for azathioprine intolerance in 67 patients. Azathioprine was not used in 11 patients and 10 patients decided to stop azathioprine for personal reasons.

Methotrexate was administered orally in 115 patients, subcutaneously in three patients and an initial intramuscular induction progressing to an oral regime in 16 patients. There was no difference in the response to treatment between these different regimes. 11 patients were treated with a combination of azathioprine and methotrexate of whom one patient had developed significant neutropenia, requiring cessation of methotrexate. 45 patients developed side effects the most common of which was nausea reported in 16. Other side effects in decreasing order of frequency were transient LFT abnormalities in 10, alopecia in one, abdominal pain in one haematological abnormalities including neutropenia in four, skin rash in three, muscle and joint pains in three, minor infections in four, and mucositis in two patients. 21 patients were withdrawn from therapy as a direct consequence of their side-effect. 94 patients received folate supplements and 39 patients, had never been on folate. No association was found between side effects and folate supplementation

187 patients completed at least three months of methotrexate therapy and were included in the group for analysis of treatment efficacy. Of these, 97 patients responded to treatment (51%). Response was defined as successful withdrawal of steroids in the steroid dependent group or the clear documentation of treatment efficacy in those who were not on steroids. All other patients were classified as treatment failures.

75 patients underwent surgical treatment. This included stricturoplasty, ileostomy, pan procto colectomy, and colectomy.

### Azathioprine Group

The azathioprine group was recruited and follow prospectively for assessment of drug response and side effects. Of the 140 patients recruited 40 withdrew due to intolerable side-effects. 100 patients completed the minimum of three months of treatment and were included for further analysis. Of these 50 patients were identified as responders to therapy, based on successful steroid withdrawal and a reduction in the Harvey Bradshaw index to less than four. 50 patients had ulcerative colitis and 49 had Crohn's disease. A small number of patients had incomplete response to treatment and have been classified as non-responders for the purpose of the study. All patients were Caucasian. TPMT genotype was available on all patients, and did not correlate with response to therapy or side effects.

TABLE 1
The effect of an 14 bp ins/del polymorphism
in HLA-G on Response to methotrexate Therapy
Allelic Frequencies

| Outcome | Insertion Allele | Deletion allele | Fisher's exact test |
|---|---|---|---|
| Response | 69 (37%) | 113 (62%) | p = 0.0061 |
| Failure | 96 (52%) | 86 (47%) | OR = 1.8 |
| | | | 95% CI: 1.2-2.7 |
| Controls | 456 (45%) | 548 (55%) | |

Genotype Frequencies

Insertion

| Outcome | yes | no | Fisher's exact test |
|---|---|---|---|
| Response | 51 (56%) | 40 (44%) | p = 0.004 |
| (91 pts.) | | | OR = 2.6, |
| Fail | 70 (77%) | 21 (23%%) | 95% CI: 1.39-4.92 |
| (91 pts.) | | | |

TABLE 2
The influence of the 14 bp ins/del polymorphism
on response to azathioprine in IBD

| | | | |
|---|---|---|---|
| Outcome | Insertion Allele | Deletion allele | Fisher's exact test |

Allele Frequencies All Patients

| | | | |
|---|---|---|---|
| Response | 75 (65%) | 39 (34%) | p = 0.0001 |
| Failure | 30 (36%) | 50 (64%) | OR = 3.2 |
| | | | CI = 1.7-5.8 |

Allele Frequencies in Ulcerative Colitis Patients

| | | | |
|---|---|---|---|
| Response | 21 (37%) | 45 (62%) | p = 0.002 |
| Failure | 26 (62%) | 16 (38%) | OR = 3.4 |
| | | | CI = 1.5-7.8 |

Allele Frequencies Crohn's Disease Patients

| | | | |
|---|---|---|---|
| Response | 18 (31%) | 45 (69%) | p = 0.0009 |
| Failure | 24 (63%) | 14 (37%) | OR = 4.2 |
| | | | CI = 1.8-10 |

TABLE 3
Genotype Frequencies of all patients treated with azathioprine

Insertion

| Outcome | yes | no | Fisher's exact test |
|---|---|---|---|
| Response | 32 | 50 | p = 0.03 |
| Fail | 30 | 21 | OR = 2.2, |
| | | | 95% CI: 1-4.5 |

TABLE 4
The comparison of the effect of Genotype in
Response to Azathioprine between CD and UC

Insertion

| | Outcome | yes | no | Fisher's exact test |
|---|---|---|---|---|
| CD | Response | 14 | 15 | p = 0.005 |
| | Fail | 17 | 2 | OR = 9, |
| | | | | 95% CI: 1.7-46 |
| UC | Response | 18 | 10 | p = 0.11 |
| | Fail | 18 | 3 | OR = 3, |
| | | | | CI = 0.07-1.2 |

### Conclusions

In rheumatoid arthritis is clear that methotrexate does not act simply as the antiproliferative agent for the cells responsible for joint inflammation. Leucocytes have lifespan considerably longer than the weekly intervals that which methotrexate needs to be administered to maintain a therapeutic response in inflammatory conditions. Rapid flare ups are seen when medication is discontinued. This makes an anti inflammatory effect of low dose methotrexate very likely. Furthermore, observations that folate supplementation did not negatively influence the efficacy of the folate receptor antagonist drugs such as methotrexate, provides added impetus to this hypothesis.

The HLA-G molecule interacts closely with cytokines and other mediators of inflammation expressed in acute or chronic inflammatory states. It allows for tolerisation and the maintenance of an anti-inflammatory state. The well described 14 bp ins/del polymorphism has been shown to have play an important role in many inflammatory conditions. It appears to correlate with the soluble form of HLA-G. This soluble isoforms is proposed to have anti-inflammatory effects. These experiments have shown that the tested polymorphism in HLA-G influences individual response to anti-inflammatory medication in inflammatory bowel disease. This effect is non-specific for both azathioprine and methotrexate. It is likely that the 14 by insertion in the HLA-G gene destabilises mRNA and leads to subsequent impairment in sHLA-G production. This in turn may inhibit response to immunomodulatory drug therapy in patients with Inflammatory Bowel Disease. Important differences are seen between the two drug groups. A superior response is noted for Crohn's disease patients over patients with ulcerative colitis in the azathioprine group. It is difficult to decide how the relative lack of UC patients in the MTX treated group has influenced this difference and caution is required in its interpretation. Furthermore the two groups differ in the mode of recruitment. The MTX group is multicenter and retrospective, the AZA group is single center and prospectively recruited. Such differences aside, these findings are consistent with those previously reported in MTX treated Rheumatoid arthritis patients.

### EXAMPLE 2

### HLA-G Marker

Details of the second marker, the presence or absence of a 14 bp insertion/deletion polymorphism in HLA-G are disclosed herein.

### Aldehyde Oxidase (SNP AOX3404A>G) Marker

The third marker is the presence of the coding SNP AOX3404A>G in the gene for Aldehyde Oxidase 1. The presence of G in this position (by comparison to A) is indicative of reduced response of a subject or azathioprine. Details of this marker were presented at the 2008 Meeting of the British Society for Gastroenterology on Thursday 13 Mar. 2008, Birmingham, GB: “Common polymorphism in the aldehyde oxidase gene is a marker of non-response to azathioprine therapy in inflammatory bowel disease” (M A Smith, T Marinaki, A Ansari, J Sanderson). An abstract of the presentation was published in Gut. 2008; 57: A1-A172, the contents of which are hereby incorporated by reference.

### Methods:

A cohort of 182 prospectively recruited patients starting 2 mg/kg AZA for IBD (Inflammatory Bowel Disease) was divided into responders (66), non-responders (44) and those discontinuing treatment due to side effects (72). TPMT activity was established using mass spectroscopy. The whole cohort was genotyped for the presence of the coding SNP AOX3404A>G using real-time PCR Taqman® drug metabolism genotyping assay, and confirmed in a subset by traditional sequencing. The presence of the 14 bp insertion/deletion in HLA-G exon 8 3′ untranslated region was established using traditional gel-based PCR. Analysis of responders vs. non-responders used chi-square and trend tests.

### Results:

All genetic markers were in Hardy-Weinburg equilibrium. Each factor was independently associated with non-response (AOX3404A>G p=0.025; TPMT activity>35 p=0.0005, HLA-G p=0.002). The additive effect on clinical response of having multiple adverse predictors was highly significant: p=1.7×10⁻⁶. Those with no adverse predictors had a 95% chance of responding to azathioprine, as compared to 14% in those with all adverse predictors. See FIG. 4.

1. A method for predicting the response of a disease, in a subject, to a drug providing 6-mercaptopurine said method comprising the step of:
(i) determining the presence or absence of a variant allele at a polymorphic site in a human leucocyte antigen (HLA)-G gene wherein the presence of said variant allele at said polymorphic site is indicative of clinical response or tolerance to said drug.

2. A method as claimed in claim 1 wherein said polymorphic site is within the 3′ untranslated region of the HLA-G gene.

3. A method as claimed in claim 2 wherein said polymorphic site is exon 8.

4. A method as claimed in claim 1 wherein said variant allele is a 14 base pair insertion or deletion polymorphism.

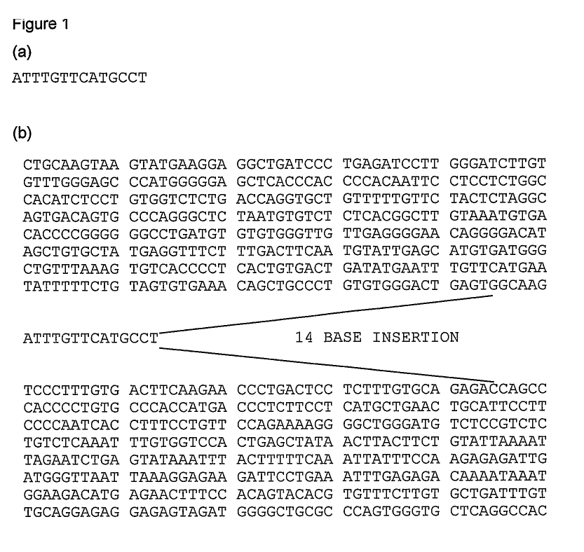

5. A method as claimed in claim 4 wherein said 14 base pair deletion/insertion polymorphism is represented by the sequence shown in FIG. 1a (SEQ ID NO: 3).

6. A method as claimed in claim 1 wherein the variant allele is of G*010101, G*0102 and/or G*010401.

7. A method as claimed in claim 1 wherein the variant allele is of G*010102, G*010103, G*0103, G*0105N and/or G*0106.

8. A method as claimed in claim 3 wherein the presence of an insertion polymorphism is indicative of an expected less favourable clinical response of the disease to said drug.

9. A method as claimed in claim 3 wherein the presence of an deletion polymorphism is indicative of an expected favourable clinical response of the disease to said drug.

10. A method as claimed in claim 1 comprising the steps of
(i) genotyping said subject at a polymorphic site in a human leucocyte antigen (HLA)-G gene; and
(ii) determining the presence or absence of a variant allele at a polymorphic site in a human leucocyte antigen (HLA)-G gene wherein the presence of said variant allele at said polymorphic site is indicative of clinical response or tolerance to said drug.

11. A method as claimed in 4 wherein the presence or absence of a deletion-insertion polymorphism is determined by determining the concentration of soluble HLA-G (sHLA-G) in a sample from a subject; decreased levels of sHLA-G being indicative of the presence of an insertion polymorphism.

12. A method as claimed in claim 1 further comprising the step of determining the presence or absence of a variant allele at a polymorphic site within a thiopurine methyltransferase (TPMT) gene wherein the presence of said variant allele at said polymorphic site is indicative of a decreased clinical tolerance, or clinical intolerance, to said drug.

13. A method as claimed in claim 12 wherein TPMT activity is measured in a biological sample from said subject and wherein either an absence, or below normal levels, of TPMT activity are indicative of an intolerance, or decreased tolerance, to said drug.

14. A method as claimed in claim 12 comprising the steps of:
(i) genotyping said subject at a polymorphic site in a TPMT gene; and
(ii) determining the presence or absence of a variant allele at said polymorphic site wherein the presence of said variant allele at said polymorphic site is indicative of a decreased clinical tolerance, or clinical intolerance, to said drug.

15. A method as claimed in claim 14 wherein the presence of a variant allele as said polymorphic site is indicative of intolerance, or decreased tolerance, to said drug.

16. A method as claimed in claim 1 further comprising the step of determining the presence or absence of a variant allele at a polymorphic site in a aldehyde oxidase (AOX) gene wherein the presence of said variant allele at said polymorphic site is indicative of a clinical response or tolerance to said drug.

17. A method as claimed in claim 16 wherein AOX activity is measured in a biological sample from said subject.

18. A method as claimed in claim 16 comprising the steps of:
(i) genotyping said subject at a polymorphic site in an AOX gene; and ii) determining the presence or absence of a variant allele at said polymorphic site wherein the presence of said variant allele at said polymorphic site is indicative of a clinical tolerance to said drug.

19. A method as claimed in claim 16 wherein the polymorphic site is in aldehyde oxidase 1 gene.

20. A method as claimed in claim 19 wherein said polymorphic site is 3404A.

21. A method as claimed in claim 20 wherein said variant allele is a SNP polymorphism.

22. A method as claimed in claim 21 wherein said SNP polymorphism is a nucleotide residue other than A.

23. A method as claimed in claim 23 wherein said SNP polymorphism is a G.

24. A method as claimed in claim 16 wherein the presence of said variant allele is indicative of a less favourable clinical response to said drug.

25. A method as claimed in claim 1 wherein the drug is selected from the group consisting of 6-mercaptopurine, azathioprine and 6-thioguanine.

26. A method as claimed in claim 25 wherein the drug is azathioprine.

27. A method for predicting the response of a disease, in a subject, to a drug providing 6-mercaptopurine said method comprising the steps of:
i) determining the presence or absence of a variant allele at a polymorphic site in a HLA-G gene and an AOX gene wherein the presence of said variant allele at said polymorphic sites is indicative of clinical response or tolerance to said drug.

28. A method as claimed in claim 27 further comprising the step of determining the presence or absence of a variant allele at a polymorphic site in a TPMT gene.

29. A method of treating or preventing disease in a subject comprising the steps of:
(i) determining the presence or absence of a variant allele at a polymorphic site in a human leucocyte antigen (HLA)-G gene; and
(ii) if said variant allele is present, administering a drug providing 6-mercaptopurine in order to prevent, delay or reduce the disease.

30. A method as claimed in claim 29 wherein the variant allele is the presence of the 14 base pair deletion polymorphism in the 3′ untranslated region of exon 8 of the HLA-G gene.

31. A method as claimed in claim 29 further comprising the step of determining the presence or absence of a variant allele at a polymorphic site in an AOX gene.

32. A method as claimed in claim 29 further comprising the step of determining the presence or absence of a variant allele at a polymorphic site in a TPMT gene.

33. A method as claimed in claim 29 wherein the disease is an immune disorder.

34. A method as claimed in claim 33 wherein the disorder is selected from an immune-mediated gastrointestinal disorder, an autoimmune disorder and graft versus host disease.

35. A method as claimed in claim 33 wherein the disorder is an inflammatory disorder selected from atopy, asthma or psoriasis or type II diabetes

36. A method as claimed in claim 33 wherein the disorder is an inflammatory joint disease selected from rheumatoid arthritis, multiple sclerosis and alkylating spondylitis.

37. A method as claimed in claim 33 wherein the disorder is lymphoblastic leukaemia.

38. A method as claimed in claim 34 wherein the disorder is an immune-mediated gastrointestinal disorder.

39. A method as claimed in claim 38 wherein the immune mediated gastrointestinal disorder is inflammatory bowel disease.

40. A kit comprising means for determining the presence or absence of a variant allele at a polymorphic site in a HLA-G gene wherein the presence of said variant allele at said polymorphic site is indicative of a clinical response or tolerance to a drug providing 6-mercaptopurine.

41. A kit as claimed in claim 40 comprising PCR primers specific for said variant allele.

42. A kit as claimed in claim 41 wherein the primers are selected from:

| | |
|---|---|
| 5′-AGCTTCACAAGAATGAGGTGGAGC-3′ | (SEQ ID NO: 1) |
| and | |
| 5′-AATGAGTCCGGGTGGGTGAGCGA-3′. | (SEQ ID NO: 2) |


SEQUENCE LISTING (5 sequences)

SEQ ID NO: 1 - DNA, 24 bp, Homo sapiens
agcttcacaagaatgaggtggagc

SEQ ID NO: 2 - DNA, 23 bp, Homo sapiens
aatgagtccgggtgggtgagcga

SEQ ID NO: 3 - DNA, 14 bp, Homo sapiens
atttgttcatgcct

SEQ ID NO: 4 - DNA, 400 bp, Homo sapiens
ctgcaagtaagtatgaaggaggctgatccctgagatccttgggatcttgtgtttgggagcccatgggggagctcacccaccccacaattcctcctctggccacatctcctgtggtctctgaccaggtgctgtttttgttctactctaggcagtgacagtgcccagggctctaatgtgtctctcacggcttgtaaatgtgacaccccggggggcctgatgtgtgtgggttgttgaggggaacaggggacatagctgtgctatgaggtttctttgacttcaatgtattgagcatgtgatgggctgtttaaagtgtcacccctcactgtgactgatatgaatttgttcatgaatatttttctgtagtgtgaaacagctgccctgtgtgggactgagtggcaag

SEQ ID NO: 5 - DNA, 400 bp, Homo sapiens
tccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaactttccacagtacacgtgtttcttgtgctgatttgttgcaggagaggagagtagatggggctgcgcccagtgggtgctcaggccac